(19) United States
(12) Patent Application Publication
(10) Pub. No.: US 2025/0005219 A1
(43) Pub. Date: Jan. 2, 2025

(54) PREDICTIVE METHOD BASED UPON MACHINE LEARNING FOR THE DEVELOPMENT OF COMPOSITES FOR TIRE TREAD COMPOUNDS

(71) Applicant: Bridgestone Europe NV/SA [BE/BE], Zaventem (BE)

(72) Inventors: Paolo Paciotta, Roma (IT); Andrea Guidi, Roma (IT); Roberto Lombardi, Roma (IT)

(21) Appl. No.: 18/709,770

(22) PCT Filed: Nov. 29, 2022
(86) PCT No.: PCT/IB2022/061517

§ 371 Date: May 13, 2024

(30) Foreign Application Priority Data
Nov. 29, 2021 (IT) 102021000030077

(51) Int. Cl. G06F 30/15; B60C 1/00; B60C 11/00

(52) U.S. Cl. CPC G06F 30/15; B60C 11/0008; B60C 1/0016; B60C 2011/0025

(57) ABSTRACT

The present invention refers to a predictive method based upon machine learning for the development of composites for tyre tread compounds.

The present invention refers to a predictive method for the viscoelastic or processability properties of rubber compounds before, during and after vulcanization, which method is based upon machine learning, to be implemented therefore by means of an electronic computer, for the development of composites for tire tread compounds.

### BACKGROUND

The present invention is in the tire manufacturing sector, in particular with reference to the determination of the composition of those rubber compounds used for manufacturing tire treads.

The RPA (rubber process analyzer), as an advanced dynamic mechanical rheological testing instrument, is generally available in every plant.

The RPA (rubber process analyzer), as an advanced dynamic mechanical rheological testing instrument, is generally available in every plant in order to monitor the production parameters of the composites during each step of the process. In fact, the workability of the composite is determined by specific ranges of descriptors of the rheometric curve and shear modulus before and after hardening, defined during the development step (e.g. ML and MH torque, T10, T50 and T90, scorch time, vulcanized and unvulcanized shear modulus G′ and tand at fixed imposed stress conditions).

These properties are ensured by the characteristics of the recipes used for the composites, in particular in terms of the ingredients, the quantity thereof and the particular synergies that are established between two or more thereof.

Commonly, the correct formulation of the recipes used for composites must go through several validation steps in the laboratory in order to first find the right technological package and then optimise the formulation by means of progressive fine-tuning until the objective is fully achieved.

Each of these iterative experimental campaigns leads, from the product point of view, to an increase in lead-times and costs in developing the product (time to market) and, from the data point of view, to the generation of a database with intrinsic variability due to random noise within the measurements made during the various test campaigns.

Prediction of product performance, in the terms outlined above, typically requires extensive laboratory testing to achieve compound validation and requires time and resources.

In particular, an object of the present invention is that of simulating laboratory tests in order to provide an accurate estimate of some of the significant viscoelastic and processability properties of composites for the production of rubber compounds for tires without the need to perform any physical tests.

The use of a software tool that can predict the behaviour of composites, and therefore tire performance, allows for:
a significant reduction in recurring costs (raw materials, labour, etc.);
optimised execution capacity and quality of laboratory tests (making it possible to allocate manpower to other activities);
shorter time to market for new products;
increased predictive precision in relation to known methodologies.

Other clear advantages over the prior art, together with the features and usages of the present invention, will become clear from the following detailed description of the preferred embodiments thereof, given purely by way of non-limiting example.

### BRIEF DESCRIPTION OF THE FIGURES

Reference will be made to the drawings in the attached figures, wherein:

FIGS. 1A and 1B show schematic block diagrams of the training steps of the machine learning and property prediction algorithm according to the present invention;

FIG. 2 shows a graph useful for verifying the time required to reach a predetermined increase in vulcanization torque.

### THEORETICAL BACKGROUND

Polymer matrix composite materials are unique materials, with both a characteristic elastic and viscous response when subjected to stress.

The prediction of the rheometric curve of the composite on the basis of one of its fundamental parameters (e.g. torque ML and MH, T10, T50 and T90, scorch time, vulcanized and unvulcanized shear modulus G′ and tand at fixed shear conditions) are fundamental for determining and evaluating the workability of the composite from mixing to extrusion and the vulcanization step, avoiding problems such as mixer downtimes, defects in the extruded product, clogging of the presses during vulcanization or under/over-vulcanization.

The processability properties are evaluated by performing the rheometric tests in multiple steps. Some of these plant-tested process parameters have recently been related to performance parameters by specific evaluation, so their prediction has become even more important for estimating plant performance variability. Such an evaluation requires several laboratory tests in order to reach validation of the composite and requires time and resources.

On the other hand, the use of a digital predictive instrument may:
Decrease recurring costs (raw materials, labour, etc. . . . );
Optimise the workload and quality of the test laboratory (allowing manpower to be concentrated on other activities);
Reduce the time to market for new products.

Therefore, potential end users are all engineers and test laboratory professionals who may benefit from the instrument.

As anticipated in the previous paragraph, the processability test apparatus is also available in plants for monitoring composites in order to comply with quality standards: the present invention has the potential to also be extended and released to plants as end users, allowing the plant technical service to evaluate changes to the characteristics of the formula in the event of formula development with a significant reduction in time to solve possible problems in plants with limited production downtime/loss, or simply improve the processability response of research and development.

The rubber process analyzer (RPA) is a valuable machine designed to measure the viscoelastic or processability properties of polymers and composites before, during and after vulcanization. The vulcanization characteristics may be determined by measuring the properties as a function of time and temperature. The test may be performed in a different condition depending on the required test method and the measurement of G′ and tand may be recorded continuously as a function of time and/or strain applied by a cyclic torque at different shear rates.

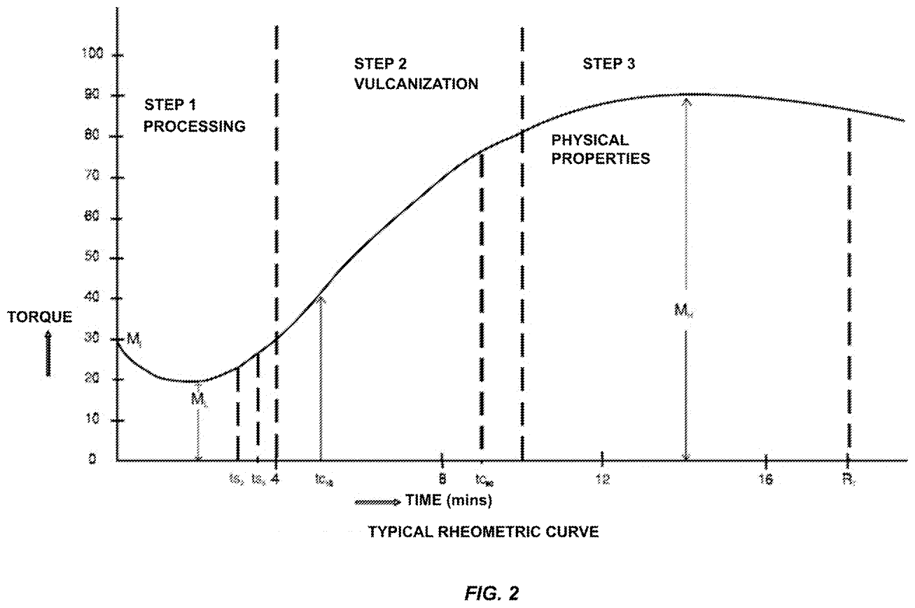

Some outputs from each test have been selected for our scope based on their cardinality in the dataset and their importance to engineers for evaluating processability:
1. ML, lower torque of the vulcanization curve, elastic modulus of green (unvulcanized) compounds;
2. MH, maximum torque of the vulcanization curve, or torque when the curve rises to the plateau, elastic modulus of the vulcanized compounds;
3. T10, T50, T90 time to reach the homologues % of ML+ΔT, where ΔT is the torque difference between MH−ML;
4. Ts, i.e. scorch time, the time to reach a set +1 dNm torque increment of the vulcanization (see FIG. 2);
5. strain G′@100% before vulcanization, which is the parameter relating to the viscosity of the green (unvulcanized) composite;
6. strain G′@1%, strain tand@15% after vulcanization, which is related to the dynamic properties of the vulcanized compound used to predict tire tread performance;
7. strain G′@50% after vulcanization, which correlates with high strain static properties.

### DETAILED DESCRIPTION OF POSSIBLE EMBODIMENTS OF THE INVENTION

The present invention will be described below with reference to the above figures.

A methodology for the prediction of viscoelastic or processability properties will therefore be described (e.g. ML and MH torque, T10, T50 and T90, scorch time (Ts), vulcanized and unvulcanized strain modulus G′ and tand at imposed strains) of composites usable for the production of rubber compounds for tires.

In general terms, a methodology for the prediction of processability properties such as ML and MH torque, T10, T50 and T90 hardening times, scorch time, shear modulus, and tand at imposed strains of new composites, never mixed before, will be described, according to the following procedure:
Generation of a database by collecting existing recipes and corresponding viscoelastic or processability properties (i.e. ML, MH, T10, T50, T90, TS, G′@100%, G′@1%, G′@50%), obtaining a primary dataset;
Procedure of integration of the primary dataset through data augmentation, to obtain an augmented dataset;
Procedure of transformation of augmented data, to obtain a transformed dataset;
Training of an algorithm based on machine learning (e.g. linear mixed model) with the transformed dataset;
Prediction, through the trained algorithm, of the viscoelastic or processability properties of new recipes to be tested.

In particular, after a dedicated “step of training the model” of a machine learning algorithm on the augmented and transformed dataset, it is possible to predict the processability properties with greater accuracy than by directly applying the algorithm to the original dataset.

In fact, in this way it is possible to drastically reduce the effect of the random noise of the database on the predictive accuracy.

In particular, after a step of training the model using the dataset contained in the augmented and transformed database as described above, it is possible to predict the viscoelastic and processability properties of the composite with greater precision than by directly applying an algorithm straight to the raw data in the database.

In fact, in this way, it is possible to drastically reduce the effect, on the predictive precision, of database noise and the intrinsic variability of the data.

This instrument is characterised by the implementation of the following actions and algorithms:
1. Collection of laboratory data representative of the recipe of the composite to be tested;
2. Data augmentation procedure: this procedure, by exploiting various techniques to introduce new characterising parameters, is able to enhance the predictive capabilities of the machine learning algorithm;
3. Procedure of transformation of augmented data: this procedure, by leveraging different transformation techniques, is able to enhance the predictive capabilities of the machine learning algorithm;
4. Machine learning algorithm (e.g. a linear mixed model algorithm): this algorithm has the purpose of predicting the processability properties of rubber compounds;

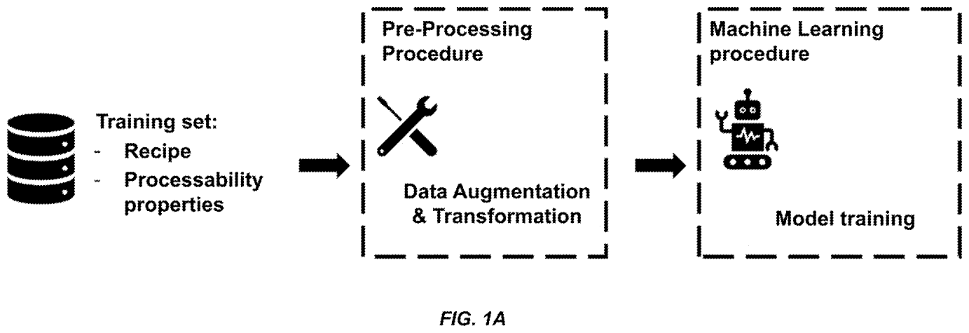

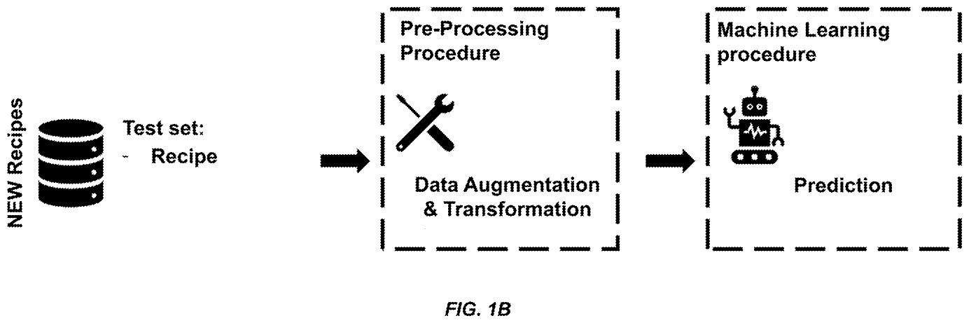

With reference to the exemplary diagrams of FIGS. 1A and 1B, the steps of a method according to the present invention will be described.

The process includes the following steps following the generation of a primary dataset, as already described:
1. Integration of data from the primary dataset (data augmentation): this procedure aims to develop new characterising parameters that must be added to the primary dataset (containing, for example, the list of ingredients and their quantities for each recipe) to give rise to an augmented dataset. This means that, for each recipe, a set of new characterising parameters is estimated in order to improve the predictive capacity of the machine learning model. The new characterising parameters, as described below, originate from the application of a set of non-homogeneous techniques, such as clustering algorithms, non-linear operators applied to input sets and some dedicated and customised artificial neural networks.
2. Transformation of the data contained in the augmented dataset:

Similar to the previous procedure, this one aims to improve predictive performance. Differently, this procedure is not focused on adding new characterising parameters, but on their transformation. As will be better described below, the adopted data transformation algorithms are based on spline functions and Box-Cox transformations.
3. Machine learning algorithm training step:

The data of the augmented and transformed dataset (recipes of compounds and corresponding characterising parameters) are provided as input to a predictive model that has been implemented through machine learning techniques (for example a mixed linear model). The internal parameters of the model are then adjusted to fit the data (training step).
4. Prediction step: once the training is completed, the algorithm is able to generalise the results and therefore it is suitable for predicting the values of the processability properties of new recipes of composites never mixed before (prediction step).

The original dataset to which the dataset integration (data augmentation) and transformation (dataset transformation) procedure has been applied is defined as a pre-processed dataset.

### Primary Dataset Data Integration Procedure:

Additional features have been put in place to improve the prediction of the composite processability properties. In fact, it has been observed that the information provided by the composite formulation is not sufficient to achieve the goal of property prediction.

For example, the mixing conditions, introduced as characterising parameters, play a significant role and have been observed as very instructive: therefore they have been used to augment the original dataset.

On the other hand, models formed by too many parameters are characterised by a lack of generalisation on the prediction. They are able to predict on the training dataset, but are not able to predict correctly on new data, i.e. operate on new test data sets. This phenomenon is called overfitting and when it occurs it makes it impossible to generate performing models in the production step.

For this reason, a technique called “data augmentation” was developed to improve predictive performance while avoiding overfitting.

The data augmentation procedure includes the estimation, and integration into the primary dataset, of one or more of the following characterising parameters:
Estimate of the Gini coefficient for each recipe: also called the Gini index or Gini ratio, it is a measure of the statistical dispersion and is applied directly to each recipe. This characterising parameter was added to provide a description of the hidden structure of the formulations.
Definition of a mixing category for each recipe: the category of the technology mixing composite recipes has been introduced as a new characterising parameter of a categorical type. This characterising parameter has been added to provide information on the mixing conditions of the ingredients (e.g. geometric parameters of the mixer itself, temperature, time and speed of the rotors) to which an effect of the physical properties that are being predicted is directed. A more comprehensive way to provide this information may be achieved by introducing all the mixing parameters directly into the datasets, but our strategy is to use only parameters of a categorical type to provide a lean solution, thus avoiding overfitting. In doing so, all the mixing parameters are summarised by a single parameter of a categorical type indicating the type and therefore the mixing category applied, such as for example a tangential or interpenetrating screw mixer.
Definition of the type of application of the composite for each recipe: the application of the composite (e.g. the component of the tire or the category of the vehicle that will mount the tire) has been introduced as a new categorical characterising parameter. This characterising parameter has been added to provide information on the final application of the composites and provide a categorical preview of the expected macro-requirements for which the composite will be developed. In doing so, all the expected macro-requisites are summarised by a parameter of a categorical type indicating the type of application for which the composite is finalised. In fact, each type of application requires different macro-requisites. Typologies of application of the composite may be for example for motor vehicles or commercial vehicles which differ from each other for example for the conditions of pressure and temperature during vulcanization.
Interaction between mixing technology and compound application type for each recipe: all the possible combinations between the mixing category and the composite application have been estimated and used to define a new characterising parameter of the categorical type for each recipe. This characterising parameter of a categorical type was introduced to explicitly describe the possible effect of the mixing conditions on the application of the composite, i.e. the macro-requirements envisaged for the composite. In fact, since the mixing category is able to have an effect on the physical properties of the composite, and the type of application of the composites provides a description of the macro-requirements of the composites, the two variables of a categorical type are connected. This connection is described through this new categorical variable which expresses all the possible combinations between the mixing category and the application of the composite.
Estimate of the total quantity of material for each recipe: it represents the sum of all the quantities of ingredients and has been estimated for each recipe. In this way the quantity of each ingredient is always related to the total quantity, i.e. different from recipe to recipe.
Ingredient ratio estimate for each recipe: an exhaustive search was implemented to determine what ratio of different ingredients correlates with the processability properties that are being predicted. Therefore, the ratios of estimated ingredients that have been shown to correlate with the target properties have been added as new characterising parameters. These characterising parameters have been added to provide an explicit description of possible non-linear ingredient terms that could be informative for predicting target properties.
Louvain grouping: the Louvain method was used to group recipes based on the co-occurrence of ingredients. In fact, Louvain's unsupervised algorithm was supplied with the recipes of the compounds that make up the available data set. As an example, the recipes may be grouped according to the presence or absence (co-occurrence) of synthetic rubber and silica rather than the co-occurrence of natural rubber and carbon black. Therefore each recipe was grouped using the Louvain method and consequently a new characterising parameter of a categorical type was estimated for the assigned cluster. The categorical parameter corresponds to the identifier of the grouping/cluster itself. This characterising parameter was introduced to provide a description of the heterogeneity of the primary dataset. In fact, the clustering algorithm is able to group different recipes based on the co-occurrence of the ingredients. This means that integrating such grouping information into the dataset may explicitly provide insight into how ingredients are used in different recipes.
K-Means grouping: each recipe was grouped using the K-Means method and consequently a characterising parameter of a categorical type was estimated for the assigned cluster. The categorical parameter corresponds to the identifier of the grouping itself. The K-Means grouping algorithm was supplied with the recipes of the composites that make up the available dataset. A grid search optimisation has been implemented to find the best hyper-parameter optimisation setting, i.e. the number of clusters to identify or the optimisation algorithm to implement. This characterising parameter was introduced to provide a description of the heterogeneity of the primary dataset. In fact, the clustering algorithm is able to group different recipes according to criteria of distance in the space of the ingredients. This means that introducing such grouping information into the primary dataset may explicitly provide insight into how ingredients are used in different recipes.
Dimensionality reduction via an autoencoder: an autoencoder (AE) is a special type of unsupervised artificial neural network trained to copy its input into its output. To achieve this, first, AE is able to map the input into a small latent space, and then AE encodes the latent representation onto the output. As a result of this operation, AE is trained to compress the data by reducing the reconstruction error. A grid search optimisation was used to find the best dimensionality of the compressed data, i.e. the dimensionality of the latent space. The so-called dimensionality reduction algorithm using the autoencoder was developed to process the set of data formed by the recipes of the composites to create a reduced-dimension, and therefore compressed, representation. The compressed data was introduced as a new characterising parameter as it represents a reduced but highly informative representation of the original recipes.

According to a preferred embodiment, all the above characterising parameters are integrated in the primary dataset.

### Transformation of Augmented Data

Prediction accuracy is vastly improved when a proper transformation operation is performed before using them for machine learning algorithm training.

The procedure of transforming the data present in the augmented dataset involves the use of one or more of the following transformation functions on the characterising ingredients and/or parameters:
B-Spline smoothing: B-Spline functions were used to smooth the numerical data of the augmented dataset before the next training step. A grid search optimisation algorithm was used to choose which ingredients and/or characterising parameters to transform.
Box-Cox transformation: the Box-Cox transformation was applied to the numerical data of the augmented dataset to make their distribution more like a normal distribution.
Scaling transformation: all numerical data of the augmented dataset were scaled to the same numerical range to improve the subsequent training step of the machine learning model.

The application of one or more of the above transformation functions leads to the generation of a transformed dataset.

According to a preferred embodiment of the present invention, all of the above transformation functions are used to process the recipes of the primary dataset.

Furthermore, according to a further embodiment, the aforementioned transformation functions, which make up the data transformation procedure, must preferably be performed following the proposed order.

According to these embodiments, all the described steps have been introduced into the pre-processing pipeline because their synergistic interaction is able to maximise the predictive performance.

The transformed (pre-processed) dataset thus obtained, after the implementation of the data augmentation and transformation procedures, may be used to start a more common training step of the machine learning model. According to an embodiment described herein, a mixed linear model was implemented and trained to deliver a predictive instrument for processability properties. Nonetheless, any other machine learning model may be used to make the final predictions as soon as the primary dataset has been processed as described.

Finally, the same pre-processing step (data augmentation and data transformation) is applied to the data relating to the recipe of the composite to be tested or to the data representative of the recipe of the composite to be tested (FIG. 1B), before being supplied as input to the already trained machine learning algorithm, in order to predict the viscoelastic or processability properties of the composite to be tested.

The present invention has heretofore been described with reference to the preferred embodiments thereof. It is intended that each of the technical features implemented in the preferred embodiments described herein, purely by way of example, can advantageously also be combined, in ways other than that described heretofore, with other features in order to give form to other embodiments which also belong to the same inventive concept and that all fall within the scope of protection afforded by the claims recited hereinafter.

1-8. (canceled)

9. A computer-implemented method for the prediction of viscoelastic or processability properties of a composite to be tested for production of tire tread compounds, the method comprising:
a) providing a primary dataset comprising recipes for already existing composites and corresponding known viscoelastic or processability properties;
b) pre-processing the primary dataset by:
i. integration of one or more characterising parameters in the primary dataset, the one or more characterising parameters being selected from among one or more of: a Gini coefficient for each recipe; a mixing category for each recipe; a type of application for each recipe; a total quantity of material for each recipe; ratios of ingredients for each recipe; Louvain grouping of said composite recipes; K-Means grouping of said composite recipes; and data reduced in dimensionality through an autoencoder applied to the data set formed by the recipes of composites,
thus obtaining an augmented dataset;
ii. transformation of the augmented dataset by applying one or more transformation functions to the ingredients and/or numerical characterising parameters of the augmented dataset, wherein the one or more transformation functions are selected from one or more of: B-Spline smoothing; Box-Cox transformation; and scaling transformation,
thus obtaining a transformed dataset;
c) training an algorithm based on machine learning using the data of the transformed dataset; and
d) applying the algorithm trained according to step (c) to a set of data that are representative of the recipe of the composite to be tested, pre-processed according to step (b), for the prediction of the viscoelastic or processability properties of the composite to be tested.

10. The method of claim 9, wherein the viscoelastic or processability properties comprise: minimum torque, maximum torque, times T10, T50 and T90, scorch time, vulcanized and unvulcanized shear modulus, and tand under imposed conditions.

11. The method of claim 9, wherein the step of integration of one or more characterising parameters in the primary dataset comprises integration of all the parameters indicated in step (i).

12. The method of claim 9, wherein the step of transformation of the augmented dataset involves application of all the transformation functions indicated in step (ii).

13. The method of claim 12, wherein the transformation functions indicated in step (ii) are performed in sequence.

14. The method of claim 13, wherein the transformation functions indicated in step (ii) are performed in an order as indicated.

15. The method of claim 9, wherein the algorithm based on machine learning is configured to apply a mixed linear model.

16. A rubber process analyzer (RPA) apparatus for the prediction of viscoelastic or processability properties of a composite to be tested for production of tire tread compounds, the apparatus configured to:
a) provide a primary dataset comprising recipes for already existing composites and corresponding known viscoelastic or processability properties;
b) pre-process the primary dataset by:
i. integration of one or more characterising parameters in the primary dataset, the one or more characterising parameters being selected from among one or more of: a Gini coefficient for each recipe; a mixing category for each recipe; a type of application for each recipe; a total quantity of material for each recipe; ratios of ingredients for each recipe; Louvain grouping of said composite recipes; K-Means grouping of said composite recipes; and data reduced in dimensionality through an autoencoder applied to the data set formed by the recipes of composites,
thus obtaining an augmented dataset;
ii. transformation of the augmented dataset by applying one or more transformation functions to the ingredients and/or numerical characterising parameters of the augmented dataset, wherein the one or more transformation functions are selected from one or more of: B-Spline smoothing; Box-Cox transformation; and scaling transformation,
thus obtaining a transformed dataset;
c) train an algorithm based on machine learning using the data of the transformed dataset; and
d) apply the algorithm trained according to (c) to a set of data that are representative of the recipe of the composite to be tested, pre-processed according to (b), for the prediction of the viscoelastic or processability properties of the composite to be tested.

17. The apparatus of claim 16, wherein the viscoelastic or processability properties comprise: minimum torque, maximum torque, times T10, T50 and T90, scorch time, vulcanized and unvulcanized shear modulus, and tand under imposed conditions.

18. The apparatus of claim 16, wherein the integration of one or more characterising parameters in the primary dataset comprises integration of all the parameters indicated in (i).

19. The apparatus of claim 16, wherein the transformation of the augmented dataset involves application of all the transformation functions indicated in (ii).

20. The apparatus of claim 19, wherein the transformation functions indicated in (ii) are performed in sequence.

21. The apparatus of claim 20, wherein the transformation functions indicated in (ii) are performed in an order as indicated.

22. The apparatus of claim 16, wherein the algorithm based on machine learning is configured to apply a mixed linear model.
